(19) United States
(12) Patent Application Publication
(10) Pub. No.: US 2013/0072915 A1
(43) Pub. Date: Mar. 21, 2013

(54) System and Method for Moving the Focal Point of a Laser Beam

(71) Applicants: Robert Edward Grant, Laguna Beach, CA (US); David Haydn Mordaunt, Los Gatos, CA (US); Kristian Hohla, Muenchen (DE); Gwillem Mosedale, Munchen (DE)

(72) Inventors: Robert Edward Grant, Laguna Beach, CA (US); David Haydn Mordaunt, Los Gatos, CA (US); Kristian Hohla, Muenchen (DE); Gwillem Mosedale, Munchen (DE)

(21) Appl. No.: 13/405,052

(22) Filed: Feb. 24, 2012

Related U.S. Application Data
(60) Provisional application No. 61/535,841, filed on Sep. 16, 2011.

(51) Int. Cl. A61F 9/011; A61B 18/20

(52) U.S. Cl. 606/4; 606/10; 606/17

(57) ABSTRACT

A system and method are provided wherein an operational characteristic of a laser beam is identified. A predetermined ophthalmic reference datum is also identified. The identified laser beam characteristic is then used in its relationship with the reference datum for guidance and control of the laser beam's focal point. In operation, the laser beam's focal point is moved through eye tissue while minimizing any deviations of the operational characteristic of the laser beam from the reference datum.

This application claims the benefit of U.S. Provisional Patent Application Ser. No. 61/535,841, filed Sep. 16, 2011

### FIELD OF THE INVENTION

The present invention pertains generally to systems and methods for performing ophthalmic laser surgery which results from the Laser Induced Optical Breakdown (LIOB) of selected tissue inside an eye. More particularly, the present invention pertains to systems and methods for performing LIOB wherein the laser beam path passes through different types of eye tissue, with each tissue type having a different threshold for LIOB. The present invention is particularly, but not exclusively, useful as a system and method for performing ophthalmic surgery wherein an operational characteristic of a laser beam is dimensionally identified, and the identified characteristic is then referenced with an imaged datum, to position the laser beam's focal point for an intended LIOB result in selected tissue of the eye.

### BACKGROUND OF THE INVENTION

Each laser beam will always have certain physical characteristics that are unique to that particular beam. In the specific case of a pulsed laser beam, apart from the wavelength of the light, laser beam characteristics will include: the location of the laser beam's focal point on the beam path; the cross section area of the laser beam at selected points (i.e. stations) along the beam path; and the energy level of the laser beam. A collective consequence of these characteristics is that each laser beam will have a determinable fluence (i.e. energy density) at each station along its path. In the case of a focused laser beam, this fluence will change inversely with changes in the beam's cross sectional area. In particular, the fluence of a focused light beam will increase as the cross sectional area of the beam decreases. Insofar as a Laser Induced Optical Breakdown (LIOB) of tissue in the eye is concerned, this change in the fluence of a laser beam can become of considerable importance.

Anatomically, it is well known that the eye has many different types of tissue, and that each of these tissue types is in direct contact with at least one other type of tissue. It is also well known that all of the various type tissues of the eye are susceptible to alteration (e.g. photoablation) by LIOB. Further, the threshold for LIOB will vary from tissue to tissue, and the occurrence of LIOB will depend on the fluence of the beam as it passes through the particular tissue. In overview, all of these factors lead to at least three separate operational considerations. For one, in an eye, the interface surface between adjacent, different type tissues is detectable by known imaging techniques, such as Optical Coherence Tomography (OCT), Scheimpflug, confocal, two-photon, laser (optical) range finding, or acoustical (non-optical) imaging. For yet another, the interface image can be used as a reference datum for laser guidance and control purposes. For yet another, it is often desirable in many ophthalmic procedures to perform LIOB on only one type of tissue, without causing collateral damage to other types of tissue.

In light of the above, it is an object of the present invention to perform Laser Induced Optical Breakdown (LIOB) on selected tissue inside an eye, wherein the laser beam path passes through different types of eye tissue. Another object of the present invention is to perform ophthalmic surgery wherein an operational characteristic of a laser beam is identified, and is referenced with a datum, to precisely position the laser beam's focal point in the eye for an intended LIOB result in selected tissue. Still another object of the present invention is to provide a laser system for performing ophthalmic surgery which is easy to use, simple to manufacture and comparatively cost effective.

### SUMMARY OF THE INVENTION

In accordance with the present invention, a system and method are provided for moving the focal point of a laser beam through eye tissue. Specifically, this is done for the purpose of performing Laser Induced Optical Breakdown (LIOB) on the tissue during a surgical procedure. As envisioned for the present invention, the path of the focal point will be through tissue that is inside the eye. Consequently, the target tissue for LIOB will be in a layer of underlying tissue, and the laser beam must necessarily pass through a layer of overlying tissue before it gets to the underlying target tissue. It typically happens that the overlying tissue and the underlying tissue will have different thresholds for LIOB. It is, nevertheless, desirable, and perhaps essential, that LIOB occur in only the target (i.e. underlying) tissue.

With this in mind, an operational concern for the present invention is that LIOB may be inadvertently performed on the overlying tissue. This is particularly problematic when the LIOB threshold of the overlying tissue is below the LIOB threshold of the underlying (target) tissue. In such a case, if the focal point of the laser beam is too close to the interface surface that is between the overlying tissue and the underlying (target) tissue, it can happen that the energy density (fluence) of the laser beam will exceed the LIOB threshold of the overlying tissue. As indicated above, this is to be avoided.

Structurally, a system in accordance with the present invention includes a laser unit for generating a laser beam with ultrashort pulses (e.g. femtosecond, picosecond or short nanosecond). Also, it includes an optical assembly for focusing the laser beam along a beam path. For example, the optical assembly may include scanners, adaptive optics, or optics with a variable numerical aperture. Importantly, this laser beam will have determinable cross sectional dimensions at respective stations along the beam path. Stated differently, depending on the energy in the laser beam, and the adaptive optics that is being used for the system, the laser beam will be dimensioned to have a determinable profile. Further, based on this profile, the energy density (i.e. fluence) of the laser beam at selected stations along the beam path can be determined.

In addition to the laser unit, the system also includes a detector for identifying a reference base inside an eye of a patient. For purposes of the present invention, this reference base is preferably an interface surface that is identified inside an eye, and is located between an overlying tissue and an underlying tissue. For purposes of the present invention, the interface surface can be established between different tissues such as the cornea/aqueous, aqueous/trabecular meshwork, aqueous/lens, lens/vitreous, corneal tissues, lens tissues and retinal tissues. As noted above, the overlying tissue will have an LIOB threshold, “T₁”, and the underlying tissue will have a different LIOB threshold, “T₂”. Preferably, the detector will be an optical device that identifies the reference base (interface surface) using any of various well known imaging techniques. More specifically, imaging techniques envisioned for the present invention include optical, interferometric and ultrasound techniques. Further, these techniques may be employed by appropriately using Optical Coherence Tomography (OCT), wavefront analysis, confocal microscopy, Scheimpflug, two-photon imaging, or laser (optical) range finding devices.

A computer is also included in the system of the present invention and it will be used for controlling an operation of the laser unit in accordance with a predetermined computer program product. Thus, the computer controls the movement of the laser beam's focal point. In particular, these movements may be in geometric and/or non-geometric patterns that include spirals, lines, rasters, circles, planes and cylinders.

The computer is also used to select a station on the beam path having a specified energy density (fluence). As envisioned for the present invention, the identification of a station involves its location on the beam path, as well as the cross sectional area of the laser beam at that location. Thus, for a beam having a particular energy, the energy density (fluence) of the beam at a particular station can be determined. This selection of a station for the present invention is important for at least two reasons. For one, the selected station will have an energy density (fluence) that is below the “T₁” LIOB threshold for the overlying tissue. For another, the selected station can be determined as being at a distance “d” upstream from the focal point of the laser beam.

An exemplary application of the present invention involves the cornea of an eye. In this example, the overlying tissue is the epithelium of an eye and the underlying surface is the stroma of the eye. Accordingly, the interface surface is against a posterior surface of the epithelium (e.g. Bowman's membrane). In one mode of operation, the computer maintains the distance “d” at a constant value in order to create a flap of stromal tissue having a substantially uniform thickness. Such a flap could be used, for example, as part of a LASIK procedure. In certain circumstances, a constant stromal thickness for such flaps, or even a predetermined stromal thickness pattern for such a flap, may be desirable in its own right. Thus, “d” can be established, according to the requirements of a particular application, to create patterns for stromal tissue that have predetermined thicknesses. For example, such applications may include LASIK procedures (as noted above), the creation of stromal pockets, and the creation of constant thickness flaps. In other modes of operation, the distance “d” can either be continuously minimized, or otherwise arbitrarily established to avoid causing unwanted LIOB. Within the eye, it will be appreciated that the interface surface may be established between any two different types of tissue. For instance, it may be established between tissues in the lens of an eye, between tissues in the retina of an eye, or between tissues in the sclera.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic of the components of a system in accordance with the present invention;

FIG. 2 is a schematic of the operational characteristics of a focused laser beam; and

FIG. 3 is an illustration of a laser beam and its focal point positioned relative to an overlying tissue layer and an underlying tissue layer during an operation of the present invention.

### DESCRIPTION OF THE PREFERRED EMBODIMENTS

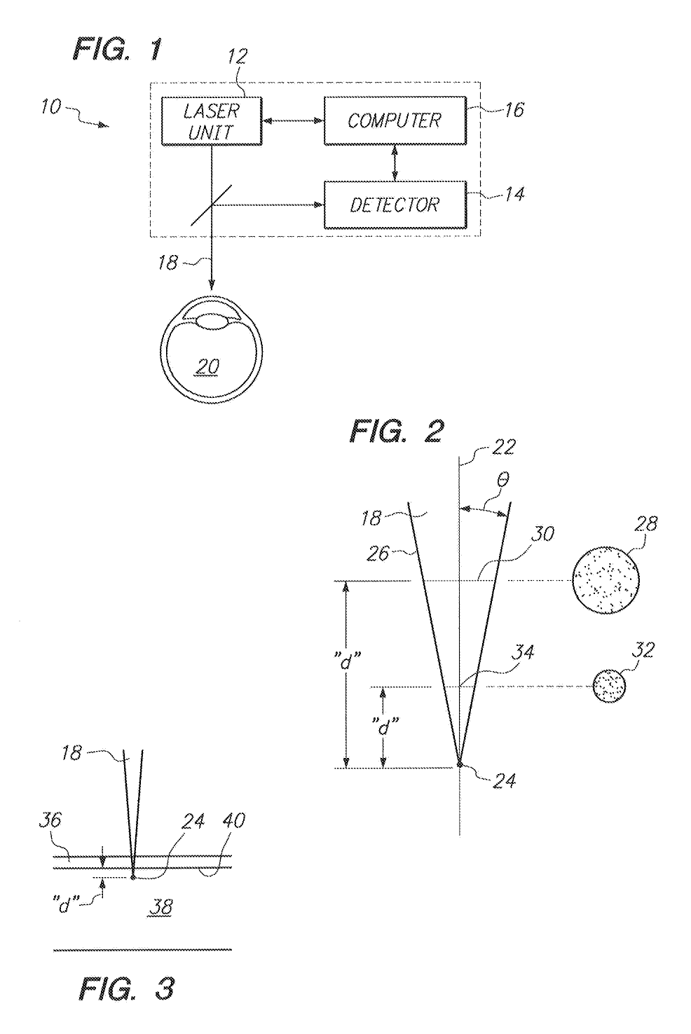

Referring initially to FIG. 1, an ophthalmic laser system in accordance with the present invention is shown and is generally designated 10. As shown, the system 10 includes a laser unit 12, a detector 14, and a computer 16. Collectively, these components of the system 10 will cooperate with each other to direct a laser beam 18 from the laser unit 12 and toward an eye 20 for the purpose of performing laser surgery on the eye 20.

For the present invention, the laser unit 12 preferably comprises what is commonly referred to as a “femtosecond laser.” Specifically, this means that the laser beam 18 which is generated by the laser unit 12 will be pulsed, and that pulses in the laser beam 18 will be of ultrashort duration (e.g. 500 fs). Further, the laser beam 18 needs to be generated with an energy level in each pulse that will cause Laser Induced Optical Breakdown (LIOB) in selected tissues of the eye 20. Thus, in general, the laser beam 18 could range from femtosecond, picosecond, or short nanosecond pulse lasers that emit their radiation in the infrared, visible, or ultraviolet wavelength range.

The detector 14 of system 10 can be a device of any type known in the pertinent art that is capable of creating two or three dimensional images of tissue structures inside the eye 20. Preferably, the detector 14 employs interferometric techniques and is an Optical Coherence Tomography (OCT) device that can create three dimensional images of interface surfaces that are identified between two different types of adjacent eye tissue.

As envisioned for system 10, the computer 16 will be connected with the laser unit 12, and with the detector 14, substantially as shown in FIG. 1. With these connections, the computer 16 uses imaging information from the detector 14, along with programmed input to the computer 16 that is provided by the operator of system 10, for moving the laser beam 18. In particular, closed loop, feedback control techniques are used by the computer 16 for the purposes of guiding and controlling the laser unit 12.

It is an important aspect of the present invention that the guidance and control of the laser unit 12 be precise, and that it be effective for the accomplishment of an intended LIOB result. In general, such control can be relatively straightforward when only homogeneous tissue is involved. There are, however, many locations in an eye 20 where LIOB may be useful, but different types of tissue are in close proximity to each other. On this point, recall that different tissues in the eye 20 have different thresholds for LIOB, and they have different refractive properties.

When considering the respective LIOB thresholds of different tissue types, the following hypothetical is helpful. If the laser beam 18 is set with an energy level that will alter one tissue having a relatively high LIOB threshold “T₂”, it is possible that another tissue with a lower LIOB threshold “T₁” can be unintentionally affected by the laser beam 18. This is particularly problematic at the interface between different tissues, and it is a situation that is obviously to be avoided. Further, it is known that beam convergence needs to be reduced the further posterior in the eye one focuses a laser beam, hence while one may avoid unintended LIOB in the corneal epithelium simply by using a highly convergent beam, this design option becomes increasingly unavailable as one moves deeper into the eye.

Apart from avoiding an unwanted outcome, the fact that different tissues in the eye 20 have different optical properties can be operationally exploited. This is so because the optical differences between adjacent tissues create an identifiable interface surface that can be located with great accuracy and precision. In particular, a detector 14 (e.g. an OCT device) that is capable of imaging the interface between different tissues in the eye 20 can provide useful information for the guidance and control of a laser unit 12. In both instances (i.e. the avoidance of unwanted tissue damage at or near a tissue interface, and the exploitation of the interface as a reference for guidance and control purposes), the operational characteristics of the laser beam 18 are important.

In FIG. 2 the laser beam 18 is shown directed along a beam path 22 to a focal point 24. In this case, when the laser beam 18 is being focused to a focal point 24, the boundary 26 of the laser beam 18 will be conically-shaped, and it will be inclined at an angle θ relative to the beam path 22. These geometric operational characteristics of the laser beam 18 can be established by the laser unit 12, as required. In the event, a consequence of this geometry is that for a given energy level in the laser beam 18, the beam 18 will have a lower fluence 28 (energy density) at an upstream station 30 on the beam path 22, and a higher fluence 32 (energy density) at a downstream station 34 on the beam path 22. Note: as used for the present invention, the word “fluence” means an energy density. In this context, the locations of stations 30 and 34 can be selected points on the beam path 22, as desired. Importantly, it will then happen that for a given value of energy in the laser beam 18, along with the location of the focal point 24 and the inclination angle “θ” of the laser beam 18, the fluence 32 at station 34 and its distance “d” from the focal point 24 can be determined. Similarly, the fluence 28 at station 30 and its distance “d” from the focal point 24 can be determined.

An exemplary application for the system 10 that involves LIOB of tissue in eye 20 is shown in FIG. 3. Specifically, FIG. 3 shows the epithelium 36 and the stroma 38 of the eye 20, with the focal point 24 of laser beam 18 positioned in the stroma 38. As shown, the focal point 24 is at a distance “d” in a posterior direction from an interface surface 40 that is identified between the epithelium 36 and the stroma 38. In this example, the stroma 38 is to be altered by LIOB. The laser beam 18, however, must first pass through the epithelium 36 and the threshold for LIOB of the epithelium 36 (T₁) is less than the threshold for LIOB of the stroma 38 (T₂) [i.e. T₂>T₁]. This then creates a situation wherein an unwanted LIOB of tissue in the epithelium 36 is a possibility. Accordingly, if the fluence 32 in laser beam 18 (see FIG. 2) corresponds with the LIOB threshold (T₁) for tissue of the epithelium 36, the focal point 24 must be in the stroma 38 at or beyond the distance “d” from the interface surface 40 to avoid LIOB of the epithelium 36. Further, by using the detector 14 to monitor movements of focal point 24 in the stroma 38, the interface surface 40 can be used as a reference datum to maintain the focal point 24 at or beyond the interface surface 40 with great accuracy and precision.

For additional applications of the present invention, wherein the relative LIOB thresholds of adjacent tissues are not a concern (e.g. the LIOB threshold of upstream tissue is greater than that of the downstream tissue) the operational characteristics of the laser beam 18 can still be used for guidance and control purposes. Specifically, the fluence (e.g. fluence 28 and 32) at stations (points or locations) on the beam path 22 (e.g. stations 30 and 34) can be identified as desired. The corresponding distances “d′” and “d” can be established for operational purposes. Again, the detector 14 can be used to identify a suitable reference datum (e.g. an interface surface such as the surface 40), and this reference datum can be appropriately used for guidance and control of the laser beam 18. For instance, the creation of an extremely thin flap (not shown) on the eye 20, having a substantially constant thickness, can be created by performing LIOB in the stroma 38 at the distance “d” from the reference surface 40. In the event, safety margins can be included into the distance “d”.

For implementing the above, a computer program product comprising program sections is provided for respectively: directing a laser beam 18 along a beam path 22 through an overlying tissue to a focal point 24 in the underlying tissue, wherein the overlying tissue has a threshold for Laser Induced Optical Breakdown (LIOB), “T₁”, and the underlying tissue has a threshold for LIOB, “T₂”; for identifying an interface surface 40 between the overlying tissue and the underlying tissue; and for positioning the focal point 24 of the laser beam 18 at a distance “d” from the interface surface 40, wherein T₁ is not equal to T₂ (T₁≠T₂). In its implementation, such a computer program product can be used to create flaps of substantially constant thickness (not shown) or incisions of variously intended configurations. Further, a computer program product can be used to adjust the numerical aperture of laser unit 12 to avoid LIOB in underlying tissue when T₂ is greater than T₁.

While the particular System and Method for Moving the Focal Point of a Laser Beam as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for positioning the focal point of a laser beam in underlying tissue which comprises:
a laser unit for generating a laser beam, and for directing the laser beam along a beam path through an overlying tissue to a focal point in the underlying tissue, wherein the overlying tissue has a threshold for Laser Induced Optical Breakdown (LIOB), “T₁”, and the underlying tissue has a threshold for LIOB, “T₂”, and wherein T₁ is not equal to T₂ (T₁≠T₂);
a detector for identifying an interface surface between the overlying tissue and the underlying tissue; and
a computer connected to the laser unit, and to the detector, for positioning the focal point of the laser beam beyond a distance “d” from the interface surface, wherein an energy density in the laser beam at the interface surface is below T₁.

2. A system as recited in claim 1 wherein the detector is used to image the interface surface using interferometric techniques.

3. A system as recited in claim 1 wherein T₂ is greater than T₁.

4. A system as recited in claim 1 wherein the overlying tissue is the epithelium of an eye and the underlying surface is the stroma of the eye, wherein the interface surface is against a posterior surface of the epithelium, and wherein the computer maintains “d” at a constant value to create a flap of stromal tissue having a substantially uniform thickness.

5. A system as recited in claim 1 wherein the distance “d” is equal to zero.

6. A system as recited in claim 1 wherein the interface surface is established between tissues in the eye selected from a group comprising the cornea/aqueous, aqueous/trabecular meshwork, aqueous/lens, lens/vitreous, corneal tissues, lens tissues and retinal tissues.

7. A system for moving the focal point of a laser beam which comprises:
a laser unit for generating a laser beam;
an optical assembly included with the laser unit for focusing the laser beam along a beam path, wherein the laser beam has predetermined cross sectional dimensions at respective stations along the beam path;
a computer for selecting a station on the beam path having a specified fluence, wherein the selected station is at a distance “d” upstream from the focal point of the laser beam;
a detector for identifying a reference base; and
a guidance unit included with the laser unit and responsive to the computer for guiding the selected station of the laser beam relative to the reference base to move the focal point of the laser beam.

8. A system as recited in claim 7 wherein the reference base is an interface surface and is identified inside an eye between an overlying tissue and an underlying tissue, wherein the overlying tissue has a threshold for Laser Induced Optical Breakdown (LIOB), “T₁”, and the underlying tissue has a threshold for LIOB, “T₂”, wherein the focal point of the laser beam is located inside the underlying tissue and the energy density of the laser beam at the interface surface is less than T₁.

9. A system as recited in claim 8 wherein the overlying tissue is the epithelium of an eye and the underlying tissue is the stroma of the eye, wherein the interface surface is against a posterior surface of the epithelium, and wherein the computer maintains the distance “d” at a constant value to create a flap of stromal tissue having a substantially uniform thickness.

10. A system as recited in claim 8 wherein the distance “d” is zero.

11. A system as recited in claim 8 wherein the interface surface is established between tissues in an eye selected from a group comprising the cornea/aqueous, aqueous/trabecular meshwork, aqueous/lens, lens/vitreous, corneal tissues, lens tissues and retinal tissues.

12. A method for using a computer program product to focus a laser beam to a focal point comprising the steps of:
directing a laser beam along a beam path;
focusing the laser beam to a focal point;
determining an energy density of the laser beam on the beam path at a selected station on the beam path;
calculating a distance “d”, wherein the distance “d” is measured along the beam path between the selected station and the focal point;
identifying a reference base; and
moving the selected station of the laser beam relative to the reference base to maintain the focal point beyond the distance “d” from the reference base.

13. A method as recited in claim 12 wherein the reference base is an interface surface and is identified inside an eye between an overlying tissue and an underlying tissue, wherein the overlying tissue has a threshold for Laser Induced Optical Breakdown (LIOB), “T₁”, and the underlying tissue has a threshold for LIOB, “T₂”, wherein the focal point of the laser beam is located inside the underlying tissue and the energy density of the laser beam at the interface surface is less than T₁.

14. A method as recited in claim 13 wherein the overlying tissue is the epithelium of an eye and the underlying surface is the stroma of the eye, wherein the interface surface is against a posterior surface of the epithelium, and wherein the method further comprises the step of maintaining the distance “d” at a constant value to create a flap of stromal tissue having a substantially uniform thickness.

15. A method as recited in claim 13 further comprising the step of minimizing the distance “d”.

16. A method as recited in claim 13 wherein the identifying step is accomplished using interferometric techniques.

17. A method as recited in claim 13 wherein the laser beam is a femtosecond laser beam.

18. A system for moving the focal point of a laser beam through the stromal tissue of an eye which comprises:
a laser unit for generating a laser beam;
an optical assembly included with the laser unit for focusing the laser beam along a beam path, wherein the laser beam has predetermined cross sectional dimensions at respective stations along the beam path;
a computer for selecting a station on the beam path at a distance “d” upstream from the focal point of the laser beam;
a detector for identifying a reference base, wherein the reference base is an interface surface between the stroma and the epithelium of the eye; and
a guidance unit included with the laser unit and responsive to the computer for guiding the selected station of the laser beam relative to the reference base to move the focal point of the laser beam through the stroma, wherein the computer maintains the focal point of the laser beam at a same distance “d” from the interface surface to create a flap of stromal tissue having a substantially constant thickness.

19. A method for using a computer program product to focus a laser beam to a focal point in the stroma of an eye, the method comprising the steps of:
directing a laser beam along a beam path;
focusing the laser beam to a focal point;
selecting a station on the beam path;
calculating a distance “d”, wherein the distance “d” is measured along the beam path between the selected station and the focal point;
identifying a reference base, wherein the reference base is an interface surface between the stroma and the epithelium of the eye; and
moving the laser beam relative to the reference base to maintain the focal point at a same distance “d” from the reference datum to create a flap of stromal tissue having a substantially constant thickness.

20. A computer program product comprising program sections for respectively: directing a laser beam along a beam path through an overlying tissue to a focal point in the underlying tissue, wherein the overlying tissue has a threshold for Laser Induced Optical Breakdown (LIOB), “T₁”, and the underlying tissue has a threshold for LIOB, “T₂”; for identifying an interface surface between the overlying tissue and the underlying tissue; and for positioning the focal point of the laser beam at a distance “d” from the interface surface, wherein T₁ is not equal to T₂ (T₁≠T₂).

21. A computer program product as recited in claim 20 wherein an energy density in the laser beam at the interface surface is below T₁.

22. A computer program product as recited in claim 20 wherein the overlying tissue is the epithelium of an eye and the underlying surface is the stroma of the eye, wherein the interface surface is against a posterior surface of the epithelium, and wherein the method further comprises the step of maintaining the distance “d” at a constant value to create a flap of stromal tissue having a substantially uniform thickness.
